(19) United States
(12) Patent Application Publication
(10) Pub. No.: US 2025/0186210 A1
(43) Pub. Date: Jun. 12, 2025

(54) FILLABLE CARDIAC ASSIST IMPLANTS

(71) Applicant: EDWARDS LIFESCIENCES CORPORATION, Irvine, CA (US)

(72) Inventors: Ilan Tamir, Irvine, CA (US); Nasser William Saleh, Stockton, CA (US)

(73) Assignee: EDWARDS LIFESCIENCES CORPORATION, Irvine, CA (US)

(21) Appl. No.: 19/059,069

(22) Filed: Feb. 20, 2025

Related U.S. Application Data
(63) Continuation of application No. PCT/US2023/074172, filed on Sep. 14, 2023.
(60) Provisional application No. 63/376,028, filed on Sep. 16, 2022.

(51) Int. Cl. A61F 2/24

(52) U.S. Cl. CPC A61F 2/2481; A61F 2220/0008; A61F 2230/0006; A61F 2230/0071

(57) ABSTRACT

A medical implant for assisting contraction of a cardiac chamber includes an implant body sized for placement at least partially within a pericardial cavity of a heart. The implant body has an opening for inflation with a filling and being flexible to expand in response to inflation with the filling. The medical implant also includes a valve disposed within the opening to allow uni-directional flow into the implant body and provide an access point for injection of the filling.

### RELATED APPLICATIONS

This application is a continuation of International Patent Application No. PCT/US2023/074172, filed Sep. 14, 2023, which claims the benefit of U.S. Provisional Application No. 63/376,028, filed Sep. 16, 2022, the disclosures of which are hereby incorporated by reference in their entirety.

### BACKGROUND

### Field

The present disclosure generally relates to the field of a medical implant devices.

### Description of Related Art

Various medical procedures involve the implantation of medical implant devices within the anatomy of the heart. Systolic heart failure can involve a reduced heart capacity to contract and/or expand.

### SUMMARY

Described herein are one or more methods and/or devices to facilitate contraction and/or expansion of the heart, particularly for patients experiencing systolic heart failure.

For purposes of summarizing the disclosure, certain aspects, advantages, and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loudspeakers, headphones, pressure transducers, temperature transducers, or using any combination of suitable technologies.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 illustrates an example representation of a human heart in accordance with one or more examples.

FIG. 2 provides a close-up view of a ventricle wall of a left ventricle of a heart.

FIG. 3 illustrates an example implant disposed within a pericardial cavity and/or other area around a chamber (e.g., left ventricle and/or right ventricle) of a heart, in accordance with one or more examples.

FIG. 4 illustrates an example implant configured to assist function of a left ventricle, right ventricle, and/or other chamber of a heart in accordance with one or more examples.

FIG. 5 illustrates an example implant configured to assist function of a left ventricle, right ventricle, and/or other chamber of a heart in accordance with one or more examples.

FIG. 6 (FIGS. 6-1 and 6-2) provides a flowchart for an example process for assisting cardiac contraction and/or expansion, in accordance with one or more examples.

FIG. 7 (FIGS. 7-1 and 7-2) provides images corresponding to steps of the process of FIG. 6.

### DETAILED DESCRIPTION

The headings provided herein are for convenience only and do not necessarily affect the scope or meaning of the claimed invention.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Certain standard anatomical terms of location are used herein to refer to the anatomy of animals, and namely humans, with respect to the preferred examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

The present disclosure relates to systems, devices, and methods for assisting heart performance and/or mitigating effects of heart failure. Implants described herein can be used to treat patients suffering from, for example, heart failure with reduced ejection fraction (HFrEF), also called systolic heart failure. Systolic heart failure can be characterized by a loss of ability of the left ventricle to contract normally. As a result, the heart may become unable to pump with enough force to push enough blood into circulation. There can be numerous causes for systolic heart failure. In some cases, systolic heart failure can be related to coronary artery disease and/or prior myocardial infarctions. This entity is termed an “ischemic cardiomyopathy” and accounts for approximately half of systolic heart failure cases.

The various implants and/or methods described herein can provide for passively (e.g., not required external power and/or force) supporting contraction and/or expansion of the left ventricle by utilizing expansion force of the diastole phase to support contraction of the heart during systole.

Some methods for assisting left ventricle performance can require one or more implants inside the heart (e.g., inside the left ventricle). Examples described herein advantageously provide for implant configured for placement outside the heart and/or with no blood contact, which can eliminate and/or minimize risks of injury and/or tissue damage. Some examples allow for targeting specific areas of the heart (e.g., infarctions and/or damaged tissue within a ventricle wall). Example devices may not require an energy source. For example, some implants may be configured to utilize the non-compromised diastole energy to support the compromised systole phase.

In some examples, implants may be delivered percutaneously and/or via one or more catheters and/or guidewires. For example, a catheter may be delivered through a puncture point at the pericardium of the heart. One or more implants may be delivered via the catheter. In some examples, one or more filling devices (e.g., syringes) may be delivered via the same or different catheter to fill the one or more implants following delivery.

Some implants described herein may advantageously be configured for filling with one or more compressible fillings. In some examples, the fillings may be configured to compress as pressure is applied to the one or more implants as a result of heart expansion during diastole. As the heart contracts and/or enters systole, the compressed fillings may be configured to forcefully expand and/or assist with compression of the heart and/or ventricle(s).

In some examples, an implant may be at least partially composed of Nitinol and/or other shape memory alloys. For examples, an implant may comprise a Nitinol frame shape set in a spheroid, spherical, and/or expanded disc shape. During diastole, the frame may be configured to compress in response to pressure applied by the walls of the heart. During systole, the frame may be configured to forcefully return and/or elastically move towards the shape set form, thus exerting a force against the walls of the heart.

### Cardiac Physiology

The anatomy of the heart is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

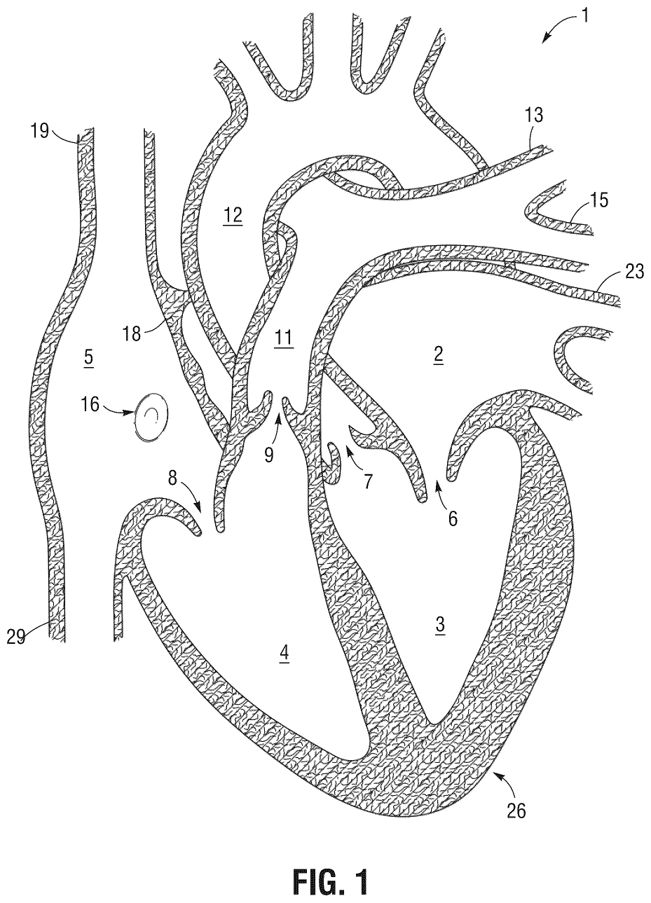

FIG. 1 illustrates an example representation of a heart 1 having various features relevant to certain examples of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery 11 via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left 15 and right 13 pulmonary arteries that branch off of the pulmonary trunk, as shown. The pulmonary veins 23 carry blood from the lungs to the left atrium 2.

In addition to the pulmonary valve 9, the heart 1 includes three additional valves for aiding the circulation of blood therein, including the tricuspid valve 8, the aortic valve 7, and the mitral valve 6. The tricuspid valve 8 separates the right atrium 5 from the right ventricle 4. The tricuspid valve 8 generally has three cusps or leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Dysfunction of a heart valve and/or associated leaflets (e.g., pulmonary valve dysfunction) can result in valve leakage and/or other health complications.

The atrioventricular (i.e., mitral and tricuspid) heart valves may further comprise a collection of chordae tendineae and papillary muscles (not shown) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae. A wall of muscle, referred to as the septum, separates the left-side chambers from the right-side chambers. In particular, an atrial septum wall portion 18 (referred to herein as the “atrial septum,” “interatrial septum,” or “septum”) separates the left atrium 2 from the right atrium 5, whereas a ventricular septum wall portion 17 (referred to herein as the “ventricular septum,” “interventricular septum,” or “septum”) separates the left ventricle 3 from the right ventricle 4. The inferior tip 26 of the heart 1 is referred to as the apex and is generally located on or near the midclavicular line, in the fifth intercostal space.

The coronary sinus 16 comprises a collection of veins joined together to form a large vessel that collects blood from the heart muscle (myocardium). The ostium of the coronary sinus, which can be guarded at least in part by a Thebesian valve in some patients, is open to the right atrium 5, as shown. The coronary sinus runs along a posterior aspect of the left atrium 2 and delivers less-oxygenated blood to the right atrium 5. The coronary sinus generally runs transversely in the left atrioventricular groove on the posterior side of the heart.

Any of several access pathways in the heart 1 may be utilized for maneuvering guidewires and catheters in and around the heart 1 to deploy implants and/or devices of the present application. For instance, access may be from above via either the subclavian vein or jugular vein into the superior vena cava (SVC) 19, right atrium 5, and from there into the coronary sinus 16. Alternatively, the access path may start in the femoral vein and through the inferior vena cava (IVC) 14 into the heart 1. Other access routes may also be used, and each can utilize a percutaneous incision through which the guidewire and catheter are inserted into the vasculature, normally through a sealed introducer, and from there the physician can control the distal ends of the devices from outside the body.

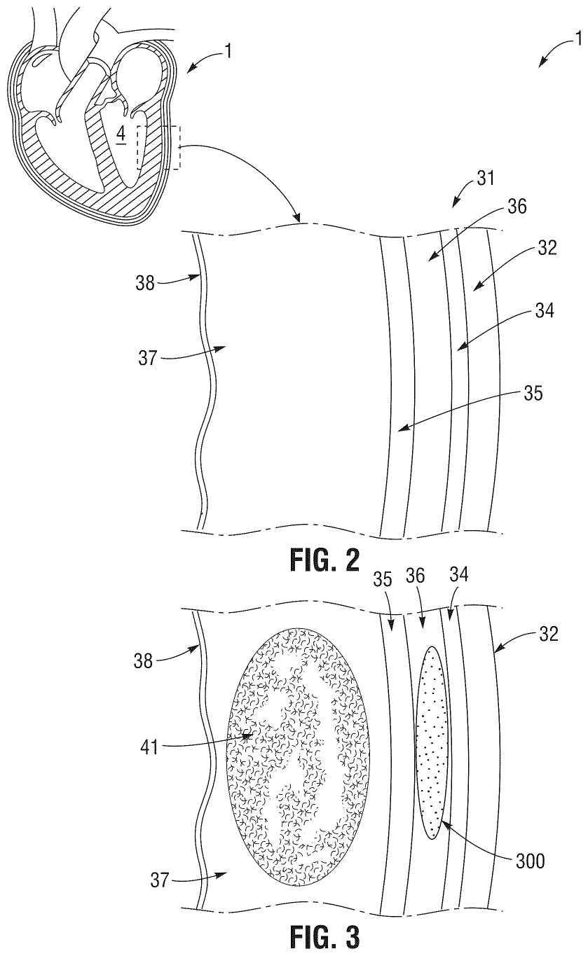

FIG. 2 provides a close-up view of a ventricle wall of a left ventricle 4 of a heart 1. The pericardium 31 is a fibrous sac that encloses the heart 1 and great vessels. The pericardium 31 keeps the heart 1 in a stable location in the mediastinum, facilitates movements of the heart 1, and separates the heart from the lungs and other mediastinal structures. The pericardium 31 also supports physiological cardiac function.

The pericardium 31 consists of two layers: the fibrous pericardium 32 and the serous pericardium 33. The fibrous pericardium 32 is a conical-shaped sac. An apex of the fibrous pericardium 32 is fused with the roots of the great vessels at the base of the heart 1. A broad base of the fibrous pericardium 32 overlies the central fibrous area of the diaphragm with which it is fused. Weak sterno-pericardial ligaments connect the anterior aspect of the fibrous pericardium 32 to the sternum.

The serous pericardium includes the parietal layer 34, which is a layer of serosa that lines the fibrous pericardium 32. The serous pericardium also includes the visceral layer 35, which is reflected around the roots of the great vessels to cover the entire surface of the heart 1. Between the parietal layer 34 and the visceral layer 35 is the pericardial cavity 36, which is a potential space that may be filled with a small amount of fluid. The part of the visceral layer 35 that covers the heart 1, but not the great vessels, is called the epicardium.

The myocardium 37 is a muscular layer of the heart. It consists of cardiac muscle cells (cardiac myocytes [also known as cardiac rhabdomyocytes] or cardiomyocytes) arranged in overlapping spiral patterns. These sheets of cells are anchored to the fibrous skeleton of the heart 1, which surrounds the atrioventricular valves and the origins of the aorta and pulmonary artery. The myocardial thickness is related to the pressure present in each chamber. The atria have thin walls and the ventricles are thicker. In adult animals, the thickness of the left ventricular free wall is approximately threefold that of the right ventricle, measured in a transverse section across the middle of the ventricles, because the pressure is greater in the systemic circulation than in the pulmonary circuit. The endocardium 38 is the innermost layer of the heart and lines the chambers and extends over projecting structures such as the valves, chordae tendineae, and papillary muscles.

Implant Devices within Pericardial Cavity

FIG. 3 illustrates an example implant 300 disposed within a pericardial cavity 36 and/or other area around a chamber (e.g., left ventricle and/or right ventricle) of a heart, in accordance with one or more examples. The implant 300 may comprise one or more compliant pads and/or similar devices. In some examples, the implant 300 may be configured to have spring-like properties and/or to transition between multiple states and/or forms. The implant 300 may utilize expansion of the left ventricle and/or chamber (e.g., during a diastole phase of the cardiac cycle) to compress and/or store energy. The implant 300 may then release this energy (e.g., during systole) to assist the contraction of the left ventricle and/or other chamber.

In some examples, the implant 300 may be filled with a filling, which can include air, low density and/or compressible polymer, foam, nitinol mesh, and/or other fillings. The implant 300 may be positioned inside the pericardial cavity 36 adjacent to one or more infarctions 41 and/or impaired sections of the left ventricle and/or chamber. The implant 300 may comprise one or more anchoring members configured to anchor to the pericardium to prevent the implant 300 from shifting inside the pericardial cavity. The implant 300 may be configured to not compress the healthy coronary arteries and/or compromise blood flow into the heart muscle. An infarction 41 can include any impaired section of tissue and/or may be present in the myocardium 37.

The implant 300 may be configured for delivery through the fibrous pericardium 32 and/or may be disposed within the pericardial cavity 36 and/or adjacent to the parietal layer 34 and/or visceral layer 35 of the pericardium. In some examples, the implant 300 may be at least partially compliant and/or compressible. The compliance of the implant 300 may be balanced such that the implant 300 may be soft enough to be compressed without interrupting the expansion of, for example, the left ventricle, during diastole and/or other cardiac stage. Moreover, the implant 300 may be sufficiently compliant to provide sufficient energy to assist with the contraction of the left ventricle and/or with the ejection of blood out of the left ventricle during systole.

In some examples, the implant 300 may be inflatable and/or may be configured to be filled with a filling, which can include one or more liquids and/or foams. The implant 300 may be configured to be filled after delivery and/or deployment within the pericardial cavity 36 and/or other target location. In some examples, the implant 300 may be disposed between the pericardium and the myocardium 37 and/or endocardium 38. In some examples, the implant 300 may comprise a support skeleton composed of one or more shape memory alloys and/or similar materials. For example, the implant 300 may comprise a Nitinol mesh support structure.

The implant 300 may be configured for percutaneous delivery and/or delivery via one or more catheters. In some examples, the implant 300 may be configured to be filled following delivery to a desired size and/or pressure.

The pericardium is made of fibrous tissue and is generally not compliant. Thus, the implant 300 may be configured to utilize the pericardium as an opposition that allows releasing of the stored energy of the implant 300 in a directional way (e.g., towards the left ventricle). In some examples, placement of the implant 300 may be done via a minimally invasive procedure and/or using a dedicated delivery system.

The implant 300 may be at least partially composed of one or more elastic materials and/or may be filled with one or more elastic materials. Such elastic materials inside the implant 300 may be pre-assembled prior to delivery and/or may be injected into the implant 300 as part of the implantation procedure (e.g., the implant 300 may be inflated with air and/or other filling).

During normal cardiac cycles, the implant 300 may be compressed between the parietal layer 34 and the visceral layer 35 due to heart expansion (e.g., during diastole). The implant 300 may be configured to store potential energy in response to compressive forces applied by the heart. The implant 300 may be configured to elastically expand and/or release stored energy towards the left ventricle and/or other chamber in response to reduced to relaxation of compression forces (e.g., during systole) to enhance the ability of the left ventricle to contract, using the pericardium as opposition.

The implant 300 may be configured to be filled with any suitable filling, which can include one or more gases (e.g., gases that are submersible in blood, which does not include air), polymer beads, and/or Nitinol. The filling may be conveyed to the implant 300 via a needle and/or tube such that the implant 300 may expand while within the heart to simplify delivery processes and/or to not require surgical delivery.

In some examples, the implant 300 may comprise a self-sealing access point through which filler is injected into the implant 300. For example, the access point may comprise a soft self-sealing polymer that seals after an injection needle and/or other delivery device is removed.

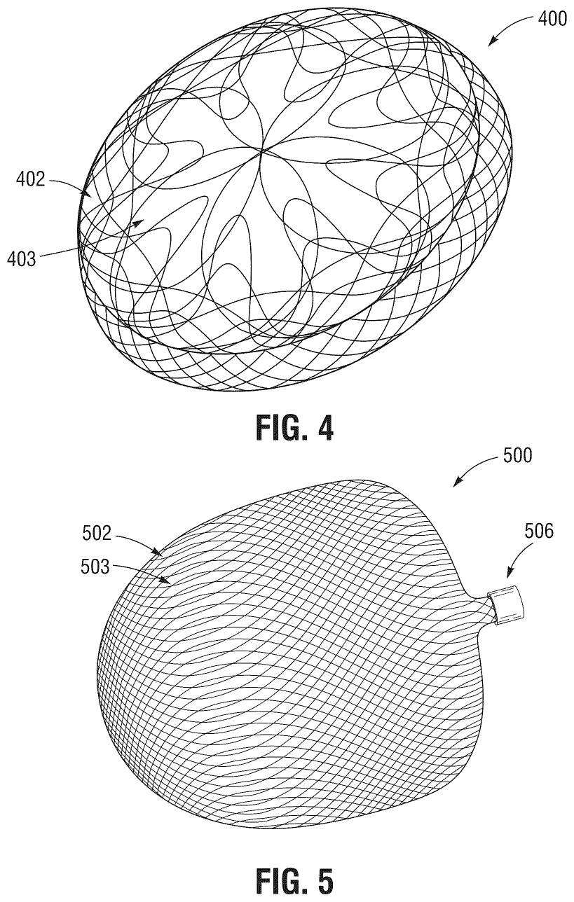

FIG. 4 illustrates an example implant 400 configured to assist function of a left ventricle, right ventricle, and/or other chamber of a heart in accordance with one or more examples. The implant 400 may be configured to provide a spacer within the pericardial cavity and/or other area within a heart. In some examples, the implant 400 may be configured for placement within a wall of the left ventricle to assist in compressing the left ventricle during systole and/or other cardiac phases.

The delivery system for the spacer device 400 may include a catheter for navigating the device 400 to the desired position. For example, the device 400 may be delivered to the implantation location in an at least partially collapsed or contracted state, wherein the device 400 may be inflated or expanded upon arrival in the implant chamber, such as a pericardial cavity. The device 400 may be inserted non-surgically in, for example, a transcatheter and/or percutaneous procedure.

In some examples, the spacer device 400 may be fixed to the pericardium and/or other tissue using one or more anchors and/or attachment members. Once positioned and/or fixed, the spacer device 400 may be expanded or inflated to a desirable size. For example, the device 400 may comprise a balloon that may be filled with fluid, such as a saline solution or a gaseous solution, through the catheter and/or syringe. In certain embodiments, the desired position of the spacer device 400 and/or the amount of expansion of the device 400 through fluid infusion or other means may be determined by the resulting compression and/or expansion (e.g., of the left ventricle) caused by the device 400.

The spacer device 400 may be fixed within the pericardial cavity and/or other location in any suitable or desirable way. For example, the device 400 may be sutured to the fibrous pericardium and/or anchored using a barb-like hook and/or corkscrew structure, or other type of anchor that may be attached to, or embedded in, the tissue of the pericardium. Placement of the spacer device 400 and/or degree of device (e.g., balloon) inflation/expansion can be guided in place using echocardiography to observe whether reduced mitral regurgitation and/or desired leaflet seating is produced thereby. It may be desirable to inject a minimal amount of fluid into the device 400 that achieves a satisfactory or desirable result with respect to compression of the left ventricle, for example. In certain embodiments, the device 400 may be injected with fluid in discrete increments (e.g., increments of between 0-10 mL or greater). By observing the performance and/or physical structure of the anatomy, contraction methods disclosed here may advantageously allow for substantially immediate or real-time adjustment of the device position and/or volume while the delivery system is locally disposed and available for adjustment operations.

Although spacer balloon devices are disclosed herein in the context of certain examples, it should be understood that assisted contraction devices in accordance with the present disclosure may comprise any desirable shape, size or type of device, and may include one or more balloons, beads, balls, strands, coils, anchors, or other devices. Furthermore, assisted contraction devices in accordance with the present disclosure may comprise expandable structure(s), such as inflatable/expandable balloons, stents, or other structures, which may be filled with any desired or practical fluid, solid or gaseous substance in various embodiments.

The implant 400 may be configured for filling with any suitable filler. In some examples, the implant 400 may be filled with one or more compressible materials. The implant 400 may be filled with one or more materials and/or may not be exclusively filled with liquids and/or non-compressible fluids. Example fillings can include gas, foam, polymers, and/or metallic forms and/or objects (e.g., Nitinol and/or other shape memory alloys), which can include metallic wires, coils, balls, and/or similar objects. In some examples, filler can include foam combined with one or more liquids and/or gases. In another example, a filler can include relatively small and/or closed-cell foam particles. A foam filler can include foam balls and/or pellets and/or elongated foam sections and/or coils.

In some examples, the implant 400 can be filled with one or more self-expanding and/or expandable materials. For example, the implant 400 may be filled with an expanding foam material configured to self-expand when injected into the implant 400. In some examples, a filler may be configured to expand and/or change characteristics when exposed to other materials. For example, the implant 400 may be filled with a foam configured to expand in response to exposure to a liquid. The implant 400 may then be filled with a liquid to cause expansion of the foam.

The implant 400 may be filled with foam particles injected with liquid. The implant 400 can be injected with fillers before, during, and/or after implantation. In some examples, the implant 400 may be filled with gases and/or biocompatible (e.g., blood compatible) materials.

In some examples, the implant 400 may be filled with one or more polymers, which can be combined with liquid and/or gas fillers. The implant 400 may be filled with one or more metals, which can be combined with liquid and/or gas fillers. In some examples, the implant 400 may comprise and/or may be filled with elongated coils. For example, the implant 400 may be filled with materials having generally solid structures and/or configured to provide compressive and/or expansive characteristics in multiple and/or different directions. The implant 400 may comprise structures configured to provide relatively high compression in parallel with the pericardium and/or heart wall and/or configured to provide relatively low compression perpendicular to the pericardium and/or heart wall.

The implant 400 may comprise a frame 402 and/or skeleton. The frame may comprise a network and/or mesh of wires, threads, fibers, and/or other materials. For example, the frame 402 may be at least partially composed of Nitinol and/or other shape memory alloys. The frame 402 may have a disc shape and/or may have a greater length/height/diameter than width.

In some examples, the implant 400 may comprise a covering 403 configured to extend across gaps of the frame 402 and/or configured to contain one or more fillings. For example, the implant 400 may comprise an air-tight and/or fluid-tight covering 403 configured to prevent leakage of one or more fillings injected into the implant 400.

The implant 400 may have a pillow-like structure and/or may be configured to deform in response to pressure to allow uncompromised flow in coronary arteries and/or other blood vessels. In some examples, the implant 400 and/or frame 402 may have a generally elastic structure and/or may be configured to naturally return to a default form (e.g., having a relatively large width) after being compressed (e.g., to a relatively small width). The implant 400 may be filled with compressible foam and/or similar materials configured to allow for a minimally invasive delivery procedure of the implant 400.

In some examples, the implant 400 may be configured to be inflated with gases and/or fluids. The frame 402 and/or covering 403 may be shape set to a predefined form such that, upon filling with any suitable filling, the implant 400 may be configured to assume the predefined form. The frame 402 and/or covering 403 may be configured to compress to a smaller form prior to and/or after filling.

The covering 403 may be composed of any suitable material. For example, the covering 403 may comprise an elastomeric balloon. The covering 403 may enclose and/or may be enclosed by the frame 402. In other examples, the implant 400 may comprise a covering 403 and/or may not comprise a frame 402. The implant 400 may be filled with polymer beads and/or any suitable filling.

In some examples, the implant 400 may comprise a valve and/or other mechanism configured to allow injection of one or more fillings into the implant 400. The valve may include a nozzle and/or any mechanism configured to provide an inflow and/or outflow passage for one or more fillings. For example, the valve may be configured to open in response to contact with a needle and/or other injector and/or may be configured to close following removal of the needle and/or other injector.

FIG. 5 illustrates an example implant 500 configured to assist function of a left ventricle, right ventricle, and/or other chamber of a heart in accordance with one or more examples. The implant 500 may be configured to provide a spacer within the pericardial cavity and/or other area within a heart. In some examples, the implant 500 may be configured for placement within a wall of the left ventricle to assist in compressing the left ventricle during systole and/or other cardiac phases.

The delivery system for the spacer device 500 may include a catheter for navigating the device 500 to the desired position. For example, the device 500 may be delivered to the implantation location in an at least partially collapsed or contracted state, wherein the device 500 may be inflated or expanded upon arrival in the implant chamber, such as a pericardial cavity. The device 500 may be inserted non-surgically in, for example, a transcatheter and/or percutaneous procedure.

In some examples, the spacer device 500 may be fixed to the pericardium and/or other tissue using one or more anchors and/or attachment members. Once positioned and/or fixed, the spacer device 500 may be expanded or inflated to a desirable size. For example, the device 500 may comprise a balloon that may be filled with fluid, such as a saline solution or a gaseous solution, through the catheter and/or syringe. In certain embodiments, the desired position of the spacer device 500 and/or the amount of expansion of the device 500 through fluid infusion or other means may be determined by the resulting compression and/or expansion (e.g., of the left ventricle) caused by the device 500.

The spacer device 500 may be fixed within the pericardial cavity and/or other location in any suitable or desirable way. For example, the device 500 may be sutured to the fibrous pericardium and/or anchored using a barb-like hook and/or corkscrew structure, or other type of anchor that may be attached to, or embedded in, the tissue of the pericardium. Placement of the spacer device 500 and/or degree of device (e.g., balloon) inflation/expansion can be guided in place using echocardiography to observe whether reduced mitral regurgitation and/or desired leaflet seating is produced thereby. It may be desirable to inject a minimal amount of fluid into the device 500 that achieves a satisfactory or desirable result with respect to compression of the left ventricle, for example. In certain embodiments, the device 500 may be injected with fluid in discrete increments (e.g., increments of between 0-10 mL or greater). By observing the performance and/or physical structure of the anatomy, contraction methods disclosed here may advantageously allow for substantially immediate or real-time adjustment of the device position and/or volume while the delivery system is locally disposed and available for adjustment operations.

Although spacer balloon devices are disclosed herein in the context of certain examples, it should be understood that assisted contraction devices in accordance with the present disclosure may comprise any desirable shape, size or type of device, and may include one or more balloons, beads, balls, strands, coils, anchors, or other devices. Furthermore, assisted contraction devices in accordance with the present disclosure may comprise expandable structure(s), such as inflatable/expandable balloons, stents, or other structures, which may be filled with any desired or practical fluid, solid or gaseous substance in various embodiments.

The implant 500 may be configured for filling with any suitable filler. In some examples, the implant 500 may be filled with one or more compressible materials. The implant 500 may be filled with one or more materials and/or may not be exclusively filled with liquids and/or non-compressible fluids. Example fillings can include gas, foam, polymers, and/or metallic forms and/or objects (e.g., Nitinol and/or other shape memory alloys). In some examples, filler can include foam combined with one or more liquids and/or gases. In another example, a filler can include relatively small and/or closed-cell foam particles. A foam filler can include foam balls and/or pellets and/or elongated foam sections and/or coils.

In some examples, the implant 500 can be filled with one or more self-expanding and/or expandable materials. For example, the implant 500 may be filled with an expanding foam material configured to self-expand when injected into the implant 500. In some examples, a filler may be configured to expand and/or change characteristics when exposed to other materials. For example, the implant 500 may be filled with a foam configured to expand in response to exposure to a liquid. The implant 500 may then be filled with a liquid to cause expansion of the foam.

The implant 500 may be filled with foam particles injected with liquid. The implant 500 can be injected with fillers before, during, and/or after implantation. In some examples, the implant 500 may be filled with gases and/or biocompatible (e.g., blood compatible) materials.

In some examples, the implant 500 may be filled with one or more polymers, which can be combined with liquid and/or gas fillers. The implant 500 may be filled with one or more metals, which can be combined with liquid and/or gas fillers. In some examples, the implant 500 may comprise and/or may be filled with elongated coils. For example, the implant 500 may be filled with materials having generally solid structures and/or configured to provide compressive and/or expansive characteristics in multiple and/or different directions. The implant 500 may comprise structures configured to provide relatively high compression in parallel with the pericardium and/or heart wall and/or configured to provide relatively low compression perpendicular to the pericardium and/or heart wall.

The implant 500 may comprise a frame 502 and/or skeleton. The frame may comprise a network and/or mesh of wires, threads, fibers, and/or other materials. For example, the frame 502 may be at least partially composed of Nitinol and/or other shape memory alloys. The frame 502 may have a disc shape and/or may have a greater length/height/diameter than width.

In some examples, the implant 500 may comprise a covering 503 configured to extend across gaps of the frame 502 and/or configured to contain one or more fillings. For example, the implant 500 may comprise an air-tight and/or fluid-tight covering 503 configured to prevent leakage of one or more fillings injected into the implant 500.

The implant 500 may have a pillow-like structure and/or may be configured to deform in response to pressure to allow uncompromised flow in coronary arteries and/or other blood vessels. In some examples, the implant 500 and/or frame 502 may have a generally elastic structure and/or may be configured to naturally return to a default form (e.g., having a relatively large width) after being compressed (e.g., to a relatively small width). The implant 500 may be filled with compressible foam and/or similar materials configured to allow for a minimally invasive delivery procedure of the implant 500.

In some examples, the implant 500 may be configured to be inflated with gases and/or fluids. The frame 502 and/or covering 503 may be shape set to a predefined form such that, upon filling with any suitable filling, the implant 500 may be configured to assume the predefined form. The frame 502 and/or covering 503 may be configured to compress to a smaller form prior to and/or after filling.

The covering 503 may be composed of any suitable material. For example, the covering 503 may comprise an elastomeric balloon. The covering 503 may enclose and/or may be enclosed by the frame 502. In other examples, the implant 500 may comprise a covering 503 and/or may not comprise a frame 502. The implant 500 may be filled with polymer beads and/or any suitable filling.

In some examples, the implant 500 may comprise a valve 506 and/or other mechanism configured to allow injection of one or more fillings into the implant 500. The valve 506 may include a nozzle and/or any mechanism configured to provide an inflow and/or outflow passage for one or more fillings. For example, the valve 506 may be configured to open in response to contact with a needle and/or other injector and/or may be configured to close following removal of the needle and/or other injector.

The valve 506 may comprise a soft sealing valve, for example a low durometer silicone rod. In some examples, the valve 506 may operate similarly to a Tuohy Borst adapter and/or similar device. The valve 506 may be configured to facilitate introduction of the catheter 701 while minimizing risk of backflow.

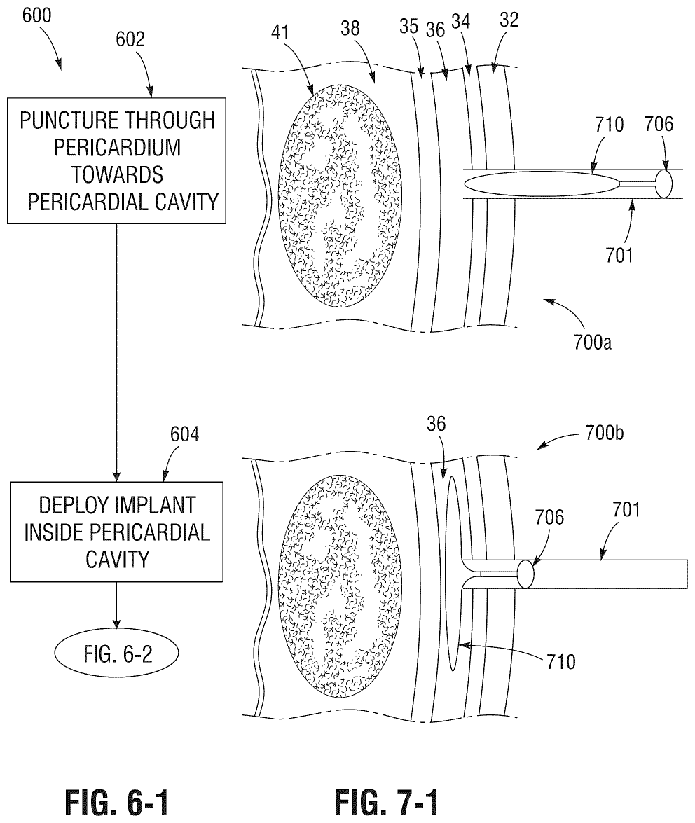

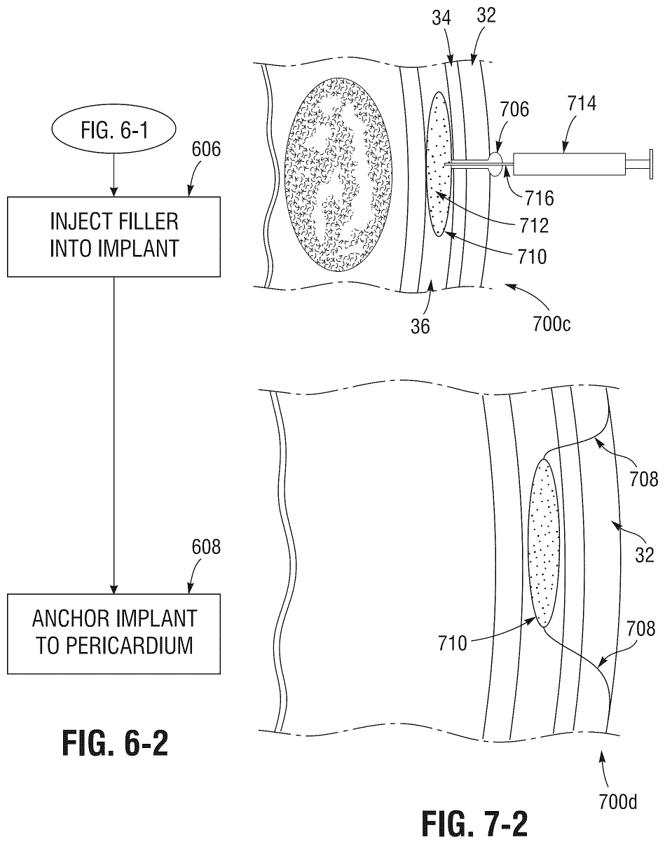

FIG. 6 (FIGS. 6-1 and 6-2) provides a flowchart for an example process 600 for assisting cardiac contraction and/or expansion, in accordance with one or more examples. FIG. 7 (FIGS. 7-1 and 7-2) provides images corresponding to steps of the process 600 of FIG. 6.

At step 602, the process 600 involves puncturing and/or extending a catheter 701 through the pericardium (e.g., through the fibrous pericardium 32, parietal layer 34 of the pericardium, and/or the visceral layer 35 of the pericardium) towards the pericardial cavity 36, as shown in image 700a of FIG. 7. In some examples the pericardium may be puncture using a puncture tool prior to delivery of the catheter 701 through the pericardium. However, the catheter may comprise a puncture tool and/or may be delivered in conjunction with a puncture tool. Alternatively, the implant 710 may be introduced into the desired location during an open-chest surgical procedure, or using other surgical or non-surgical techniques known in the art.

A puncture point of the pericardium may be at or near one or more infarctions 41 and/or damaged areas of tissue within, for example, the endocardium 38. For example, one or more implants 710 may be delivered adjacent to one or more infarctions 41 to facilitate treatment of the infarctions 41 and/or to maximize effectiveness of the implants 710.

The implant 710 may comprise a compliant pillow pressurized with gas, low density polymer(s), foam, and/or any suitable fillers. The implant 710 may be in an at least partially deflated form during delivery to the pericardial cavity 36.

In some examples, the implant 710 may comprise a soft sealing valve 706, for example a low durometer silicone rod. In some examples, the valve 506 may operate similarly to a Tuohy Borst adapter and/or similar device. The valve 506 may be configured to facilitate introduction of the catheter 701 while minimizing risk of backflow.

At step 604, the process 600 involves deploying the implant 710 at least partially within the pericardial cavity 36 and/or other location, as illustrated in image 700b of FIG. 7. The implant 710 may be extended beyond a distal end of the catheter 701 to deploy the implant 710 into the pericardial cavity 36.

At step 606, the process 600 involves injecting one or more fillers 712 into the implant 710 using one or more injectors 714, as illustrated in image 700c of FIG. 7. An injector 714 can include a syringe and/or similar device. At least a portion of the injector 714 (e.g., a needle 716) may be configured to extend through the valve 706 and/or into the implant 710. The implant 710 may be filled following delivery into the pericardial cavity 36. In some examples, the injector 714 may be configured to extend through the fibrous pericardium 32 and/or parietal layer 34 of the pericardium to reach the implant 710.

The valve 706 may be configured to extend out of the pericardial cavity 36 and/or outside the heart (e.g., against an exterior of the fibrous pericardium 32). The valve 706 may be configured to provide a hermetic septum seal to minimize and/or eliminate backflow from the implant 710. In some examples, the injector 714 may comprise luer terminations that comply with EN/ISO standards.

In some examples, the implant 710 may comprise one or more side ports for delivery medication. The valve 706 may remain attached to the implant 710 following delivery and/or may be removed. In some examples, the valve 706 may be configured to provide an anchor for the implant 710. The valve 706 may protrude at least partially out of the pericardial cavity 36 and/or pericardium and/or may provide an access point for the injector 714. The valve 706 may be configured to seal the implant 710 and/or to seal an incision made in the pericardium.

The implant 710 may be filled with fluid or other substance in order to expand a volume of the implant 710 or space occupied thereby. In some examples, the implant 710 comprises a balloon or the like, wherein the balloon is filled with a saline solution or other fluid or gas. In some examples, the degree to which the pericardial cavity 36 is expanded corresponds to an amount sufficient to exert desired pressure on the left ventricle.

The amount of fluid or gas used to fill the device may be determined by the resulting movement of the left ventricle during diastole and/or systole. The effect on the left ventricle may be evaluated to determine whether a desired level of inflation is reached. In some examples, filler may be removed from the implant 710 via the injector 714 if it is determined that too much filler has been injected into the implant 710. In some examples, the operator may use echocardiography or any other suitable means to observe the movement of the left ventricle, such as in real time. The results of the device placement may be observed continuously or at selected intervals to determine when the left ventricle has been affected sufficiently to provide a desired contraction and/or expansion of the left ventricle. Therefore, the process 600 and/or other processes, devices and systems disclosed herein may advantageously provide a tunable device, which may be tuned while monitoring the effect of the device, such as through the use of echo or other visualization guidance.

While the implant 710 may be delivered and/or employed without use of additional anchors 708, additional anchors 708 may be used. At step 608, the process 600 involves anchoring the implant 710 within the pericardial cavity 36 using one or more anchoring arms 708, as shown in image 700d of FIG. 7. In some examples, the anchoring arms 708 may be configured to anchor the implant 710 to the pericardium (e.g., to the fibrous pericardium 32). While two anchoring arms 708 are shown in FIG. 7, any number of anchors 708 may be used. While the implant 710 is shown without the valve 706 in image 700d, the valve 706 may remain in place following delivery of the implant 710.

The process 600 and/or other processes, devices and systems disclosed herein may advantageously provide a mechanism for assisting cardiac contraction using a fully transcatheter procedure on a beating heart. In some examples, the implant 710 may be designed to be retrievable.

The above method(s) can be performed on a living animal or on a simulation, such as on a cadaver, cadaver heart, anthropomorphic ghost, simulator (e.g., with body parts, heart, tissue, etc. being simulated).

### Additional Description of Examples

Provided below is a list of examples, each of which may include aspects of any of the other examples disclosed herein. Furthermore, aspects of any example described above may be implemented in any of the numbered examples provided below.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Example 1: A method for treating heart failure, said method comprising delivering an implant into a pericardial cavity of a heart using a delivery system, inflating the implant with a filling to expand the implant, and releasing the implant from the delivery system.

Example 2: The method of any example herein, in particular example 1, further comprising delivering the implant via a catheter.

Example 3: The method of any example herein, in particular example 1, further comprising delivering the implant into the pericardial cavity adjacent to an infarction in a myocardium of the heart.

Example 4: The method of any example herein, in particular example 1, wherein the implant has a disc shape.

Example 5: The method of any example herein, in particular example 1, wherein the implant has a spheroid shape.

Example 6: The method of any example herein, in particular example 1, wherein the implant has a spherical shape.

Example 7: The method of any example herein, in particular example 1, wherein the implant comprises a frame and a covering.

Example 8: The method of any example herein, in particular example 7, wherein the frame comprises a network or mesh of thin wires or threads.

Example 9: The method of any example herein, in particular example 8, wherein the frame is at least partially composed of Nitinol.

Example 10: The method of any example herein, in particular example 1, wherein the implant is at least partially composed of foam.

Example 11: The method of any example herein, in particular example 1, wherein the implant comprises an inflow valve.

Example 12: The method of any example herein, in particular example 1, wherein the filling is at least partially compressible.

Example 13: The method of any example herein, in particular example 12, wherein the filling comprises one or more biocompatible gases.

Example 14: The method of any example herein, in particular example 12, wherein the filling comprises a foam.

Example 15: The method of any example herein, in particular example 14, wherein the filling also comprises one or more liquids.

Example 16: The method of any example herein, in particular example 15, wherein the foam comprises one or more closed-cell foam particles filled with the one or more liquids.

Example 17: The method of any example herein, in particular example 15, wherein the foam is configured to expand when exposed to the one or more liquids.

Example 18: The method of any example herein, in particular example 14, wherein the filling also comprises one or more gases.

Example 19: The method of any example herein, in particular example 14, wherein the filling comprises foam balls or pellets.

Example 20: The method of any example herein, in particular example 14, wherein the filling comprises elongated foam sections or coils.

Example 21: The method of any example herein, in particular example 14, wherein the foam is self-expanding.

Example 22: The method of any example herein, in particular example 12, wherein the filling comprises one or more polymers.

Example 23: The method of any example herein, in particular example 22, wherein the filling also comprises one or more liquids.

Example 24: The method of any example herein, in particular example 12, wherein the filling comprises one or more metallic objects.

Example 25: The method of any example herein, in particular example 24, wherein the filling also comprises one or more liquids.

Example 26: The method of any example herein, in particular example 1, wherein the implant comprises a self-sealing access point.

Example 27: The method of any example herein, in particular example 26, wherein the access point comprises one or more soft polymers.

Example 28: The method of any example herein, in particular example 1, further comprising anchoring the implant in place via one or more anchoring arms coupled to the implant.

Example 29: The method of any example herein, in particular example 28, wherein the one or more anchoring arms are configured to anchor to a fibrous pericardium layer of a pericardium.

Example 30: The method of any example herein, in particular example 29, wherein the one or more anchoring arms are configured to extend through a parietal layer of a pericardium.

Example 31: A medical implant comprising an implant body configured for placement at least partially within a pericardial cavity of a heart, the implant body configured to be inflated with a filling and configured to expand in response to inflation with the filling.

Example 32: The medical implant of any example herein, in particular example 31, wherein the implant body is configured for percutaneous delivery to the pericardial cavity.

Example 33: The medical implant of any example herein, in particular example 31, wherein the implant body has a disc shape.

Example 34: The medical implant of any example herein, in particular example 31, wherein the implant body has a spheroid shape.

Example 35: The medical implant of any example herein, in particular example 31, wherein the implant body has a spherical shape.

Example 36: The medical implant of any example herein, in particular example 31, wherein the implant body comprises a frame and a covering.

Example 37: The medical implant of any example herein, in particular example 36, wherein the frame comprises a network or mesh of thin wires or threads.

Example 38: The medical implant of any example herein, in particular example 37, wherein the frame is at least partially composed of Nitinol.

Example 39: The medical implant of any example herein, in particular example 31, wherein the implant body is at least partially composed of foam.

Example 40: The medical implant of any example herein, in particular example 31, wherein the implant body comprises an inflow valve.

Example 41: The medical implant of any example herein, in particular example 31, wherein the filling is at least partially compressible.

Example 42: The medical implant of any example herein, in particular example 41, wherein the filling comprises one or more biocompatible gases.

Example 43: The medical implant of any example herein, in particular example 41, wherein the filling comprises a foam.

Example 44: The medical implant of any example herein, in particular example 43, wherein the filling also comprises one or more liquids.

Example 45: The medical implant of any example herein, in particular example 44, wherein the foam comprises one or more closed-cell foam particles filled with the one or more liquids.

Example 46: The medical implant of any example herein, in particular example 44, wherein the foam is configured to expand when exposed to the one or more liquids.

Example 47: The medical implant of any example herein, in particular example 43, wherein the filling also comprises one or more gases.

Example 48: The medical implant of any example herein, in particular example 43, wherein the filling comprises foam balls or pellets.

Example 49: The medical implant of any example herein, in particular example 43, wherein the filling comprises elongated foam sections or coils.

Example 50: The medical implant of any example herein, in particular example 43, wherein the foam is self-expanding.

Example 51: The medical implant of any example herein, in particular example

41, wherein the filling comprises one or more polymers.

Example 52: The medical implant of any example herein, in particular example 51, wherein the filling also comprises one or more liquids.

Example 53: The medical implant of any example herein, in particular example 41, wherein the filling comprises one or more metallic objects.

Example 54: The medical implant of any example herein, in particular example 53, wherein the filling also comprises one or more liquids.

Example 55: The medical implant of any example herein, in particular example 31, wherein the implant body comprises a self-sealing access point.

Example 56: The medical implant of any example herein, in particular example 55, wherein the access point comprises one or more soft polymers.

Example 57: The medical implant of any example herein, in particular example 31, further comprising one or more anchoring arms coupled to the implant body and configured to anchor the implant body in place.

Example 58: The medical implant of any example herein, in particular example 57, wherein the one or more anchoring arms are configured to anchor to a fibrous pericardium layer of a pericardium.

Example 59: The medical implant of any example herein, in particular example 58, wherein the one or more anchoring arms are configured to extend through a parietal layer of a pericardium.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method for assisting contraction and expansion of a heart chamber, the method comprising:
identifying a damaged wall of the heart chamber;
deploying an implant body within a pericardial cavity adjacent to the damaged wall, the implant body having an opening for inflation; and
injecting a filling into the implant body via the opening to expand the implant body;
wherein the implant body is compliant for facilitating contraction and expansion of the heart chamber.

2. The method of claim 1, wherein the implant body comprises a network of interwoven wires that are shape-set to return to an expanded form following compression.

3. The method of claim 1, wherein the filling comprises an expandable foam.

4. The method of claim 1, wherein the implant body comprises a self-sealing valve disposed within the opening to allow uni-directional flow into the implant body and provide an access point for injection of the filling.

5. The method of claim 1, further comprising percutaneously delivering the implant body to the pericardial cavity.

6. The method of claim 1, further comprising deploying the implant body between a parietal layer and a visceral layer of the heart chamber.

7. The method of claim 1, wherein the implant body has a disc shape.

8. The method of claim 1, wherein the implant body has a spheroid shape.

9. The method of claim 1, wherein the implant body has a spherical shape.

10. The method of claim 1, wherein the implant body comprises a frame and a covering.

11. The method of claim 1, wherein the implant body comprises one or more anchoring arms, the method further comprising deploying the one or more anchoring arms through layers of the heart chamber to anchor the implant body in place.

12. A method for assisting systolic contraction of a heart chamber, the method comprising:
identifying a damaged wall of the heart chamber;
deploying an implant body at least partially within a pericardial cavity adjacent to the damaged wall of the heart chamber, the implant body having an opening for inflation and comprising one or more anchoring arms;
deploying the one or more anchoring arms through layers of the heart chamber to anchor the implant body in place; and
injecting a filling into the implant body via the opening to cause expansion of the implant body;
wherein the implant body is compliant for facilitating contraction and expansion of the heart chamber.

13. The method of claim 12, wherein the implant body comprises a network of interwoven wires that are shape-set to return to an expanded form following compression.

14. The method of claim 12, wherein the filling comprises an expandable foam.

15. The method of claim 12, further comprising deploying the implant body between a parietal layer and a visceral layer of the heart chamber.

16. A method for assisting systolic contraction of a heart chamber, the method comprising:
identifying a damaged wall of the heart chamber;
deploying an implant body at least partially within a pericardial cavity adjacent to the damaged wall of the heart chamber, the implant body having an opening for inflation and comprising a valve disposed within the opening and sized to protrude at least partially out of the pericardial cavity; and
injecting a filling into the implant body via the opening to cause expansion of the implant body;
wherein the implant body is compliant for facilitating contraction and expansion of the heart chamber.

17. The method of claim 16, wherein the implant body comprises a network of interwoven wires that are shape-set to return to an expanded form following compression.

18. The method of claim 16, wherein the filling comprises an expandable foam.

19. The method of claim 16, wherein the implant body comprises one or more anchoring arms, the method further comprising deploying the one or more anchoring arms through layers of the heart chamber to anchor the implant body in place.

20. The method of claim 16, further comprising deploying the implant body between a parietal layer and a visceral layer of the heart chamber.
